(19) United States
(12) Patent Application Publication
(10) Pub. No.: US 2004/0105886 A1
(43) Pub. Date: Jun. 3, 2004

(54) Divalproex sodium tablets

(76) Inventors: Chih-Ming Chen, Taipei (TW); Boyong Li, Morgantown, WV (US)

Correspondence Address: DAVIDSON, DAVIDSON & KAPPEL, LLC, 14th Floor, 485 Seventh Avenue, New York, NY, 10018, US

(21) Appl. No.: 10/651,805

(22) Filed: Aug. 29, 2003

Related U.S. Application Data
(63) Continuation-in-part of application No. 10/465,702, filed on Jun. 19, 2003.
Application No. 10/465,702 is a continuation of application No. 09/785,069, filed on Feb. 16, 2001, now Pat. No. 6,610,326.

(51) Int. Cl. A61K 9/20; A61K 31/19

(52) U.S. Cl. 424/465; 514/557

(57) ABSTRACT

A process for preparing divalproex sodium tablets is provided. The process comprises preparing a neutralized divalproex sodium solution by combining divalproex sodium, having a sodium valproate and a valproic acid moiety, with an aqueous solvent and a base, e.g., sodium hydroxide, the base being in sufficient amount to ensure neutralization of the valproic acid moiety of the divalproex sodium. The neutralized divalproex sodium solution is sprayed onto a pharmaceutically acceptable carrier, and processed to obtain divalproex sodium tablets.

This application is a continuation-in-part of U.S. patent application Ser. No. 10/465,702, filed on Jun. 19, 2003; which is a continuation of U.S. patent application Ser. No. 09/785,069, filed Feb. 16, 2001, now U.S. Pat. No. 6,610,326, issued on Aug. 26, 2003, the disclosures of which are hereby incorporated by reference in their entireties.

### FIELD OF THE INVENTION

The present invention is related to a process for formulating divalproex sodium solid oral dosage forms. The process comprises preparing a neutralized divalproex sodium solution, wherein the valproic acid moiety of the divalproex sodium is neutralized by addition of a strong base. The neutralized divalproex sodium solution is subsequently processed into a solid dosage form, such as divalproex sodium tablets.

### BACKGROUND OF THE INVENTION

Valproic acid, or 2-propylpentanoic acid, and its salts and derivatives are compounds with anticonvulsant properties. Of these, valproic acid and its sodium salt (sodium valproate) are the most well known. U.S. Pat. No. 3,325,361 describes the use of valproic acid, sodium valproate and other salts and derivatives of valproic acid as anti-convulsants.

It has been recognized by those skilled in the art that both valproic acid and sodium valproate are difficult to formulate into solid oral dosage forms. Valproic acid, for example, is an oily liquid. Sodium valproate is known to be very hygroscopic and to liquify rapidly, and is, therefore, difficult to formulate into tablets.

Efforts have been made to address the problems associated with formulating valproic acid and sodium valproate into solid oral dosage forms. U.S. Pat. No. 5,049,586 (Ortega, et al.) describes valproic acid tablets having a specific composition, which tablets are said to be stable. The tablets contain valproic acid, magnesium oxide, corn starch, poyvinylpyrrolidone, sodium carboxymethylcellulose, and magnesium stearate in specific proportions.

U.S. Pat. No. 5,017,613 (Aubert, et al.) describes a process for preparing a composition containing valproic acid in combination with valproate sodium, wherein the process does not use any binder or granulating solvent. In the process, a mixture of valproic acid and ethylcellulose is prepared and valproate sodium is added to the mixture to form drug granules in the absence of any binder or granulating solvent. Precipitated silica is added to the granules before the compression into tablets.

Efforts have also been made to overcome the limited utility of valproic acid and sodium valproate in formulating solid dosage forms by creating a different salt form or a derivative of valproic acid. U.S. Pat. No. 4,895,873 (Schafer) describes a crystalline calcium salt of valproic acid, in which five valproic acid radicals are associated with one calcium ion. The crystalline salt, called calcium pentavalproate, is said to be non-hygroscopic.

U.S. Pat. No. 4,558,070 (Bauer, et al.) describes potassium, cesium or rubidium salt of valproic acid, which is prepared by combining 4 moles of valproic acid with 1 mole of the potassium, cesium or rubidium. U.S. Pat. No. 4,699,927 (Deboeck) describes arginine, lysine, histidine, ornithine or glycine salts of valproic acid.

U.S. Pat. Nos. 5,212,326 and 4,988,731 (Meade) describe divalproex sodium and its preparation. Divalproex sodium is described as an ionic oligomer in which one mole each of the valproic acid form coordinate bonds with the sodium of the sodium valproate molecule, where the valproate ion is ionically bonded to the sodium ion. Meade also describes the oligomeric compound as having better physical properties than either monomer from which it is made in that the oligomer is a crystalline, non-hygroscopic, stable solid compound.

Some patents describe sustained release dosage forms for divalproex sodium, valproic acid, its salts, amides, or other derivatives. U.S. Pat. No. 5,980,943 (Ayer, et al.) describes a sustained release delivery device for administering divalproex sodium, valproic acid, and its salts and derivatives. The device comprises a semipermeable wall containing drug granules that are microencapsulated with polyalkylene oxide or carboxymethylcellulose polymer.

U.S. Pat. No. 4,913,906 (Friedman, et al.) describes a controlled release dosage form containing divalproex sodium, valproic acid, valpromide and other valproic acid salts and derivatives. The composition is prepared by mixing the drug with hydroxypropyl cellulose, ethylcellulose, or esters of acrylic and methacrylic acid, and by applying high pressure to the mixture of the ingredients.

U.S. Pat. No. 5,807,574 (Cheskin, et al.) describes a controlled release dosage form containing divalproex sodium and a process for its preparation. The process involves melting divalproex sodium and mixing it with a molten wax to form a divalproex sodium-wax composite. The drug-wax mixture is formulated into a capsule.

U.S. Pat. No. 5,169,642 (Brinker, et al.) describes a sustained release dosage form containing granules of divalproex sodium, valproic acid or amides or esters or salts thereof and a polymeric viscosity agent. The drug is coated with a sustained release composition comprising specified portions of ethylcellulose or a methacrylic methylester, plasticizer, and detactifying agent.

U.S. Pat. No. 5,068,110 (Fawzi, et al.) describes various delayed-release tablets and capsules currently marketed, including the delayed-release divalproex sodium tablets manufactured by Abbott Laboratories, and states that the stability of an enteric coated capsules is increased by the application of thicker, higher levels of the enteric coating having a thickness of 14 mg/cm² to 24 mg/cm², alone or in combination with a hydroxypropylcellulose, hydroxymethylcellulose or hydroxypropylmethyl cellulose coating.

Divalproex sodium is a oligomer having a 1:1 molar ratio of sodium valproate and valproic acid. The oligomer is described as a stable crystalline solid and is designated as sodium hydrogen bis (2-propyl pentanoate).

Upon administration, divalproex dissociates into valproate ion in the gastrointestinal tract, and in that form exerts its pharmacological effect. Divalproex sodium is indicated for the treatment of patients with complex partial seizures, as well as for the treatment of mania associated with bipolar disorders and for prophylaxis of migraine headaches.

U.S. Pat. No. 4,558,070 (Bauer, et al.) indicates that divalproex sodium is a highly stable, non-hygroscopic, crystalline compound. Bauer also discusses a theory behind the stability of divalproex sodium, stating that it is not a mixture of the two precursors but a chemical entity, and that in the oligomer, the outer shell of electrons of the sodium atom is filled by coordination to the oxygen atoms of both valproic acid and valproate ions, resulting in a stable complex where the sodium ion is completely surrounded by oxygen. Bauer, et al., therefore, appears to indicate that the particular oligomeric structure and the molar ratio of divalproex sodium accounts for the stability of the compound.

Applicants have discovered that divalproex sodium may be formulated into stable solid oral dosage forms, even in the absence of the oligomeric structure and the equimolar ratio of sodium valproate and valproic acid.

### OBJECTS AND SUMMARY OF THE INVENTION

It is an object of the invention to provide a process for preparing a divalproex sodium composition.

It is a further object of the invention to provide a process for preparing a divalproex sodium composition, wherein the process comprises preparing a neutralized divalproex sodium solution by combining divalproex sodium, having a valproic acid moiety and a sodium valproate moiety, with a base (e.g., sodium hydroxide) and an aqueous solvent. The base is added in sufficient amount to ensure neutralization of the valproic acid moiety of the divalproex sodium. In the neutralized divalproex sodium solution, divalproex sodium is not present as its oligomeric structure or the 1:1 molar ratio of sodium valproate and valproic acid. The valproic acid of the divalproex sodium is neutralized. Preferably the neutralized divalproex sodium solution contains from about 20 to about 60% valproic acid activity.

It is a further object of the invention to provide an oral solid dosage form containing a therapeutically effective amount of divalproex sodium wherein the divalproex sodium is not present as an oligomeric structure or a 1:1 molar ratio of sodium valproate to valproic acid. It is a further object to provide a new divalproate formulation which provides a delayed release of valproate ion when the dosage form is orally administered to human patients, which dosage form is bioavailable and provides a therapeutic effect which is considered bioequivalent to delayed release divalproex sodium tablets, manufactured by Abbot Laboratories (Depakote®).

The neutralized divalproex sodium solution is sprayed onto a pharmaceutically acceptable carrier, and the resulting mixture may be processed to obtain a divalproex sodium tablet.

In one embodiment of the invention, the pharmaceutically acceptable carrier comprises a plurality of particles of a material such as, for example, anhydrous lactose or microcrystalline cellulose. A granulate is formed by spraying the neutralized divalproex sodium solution onto the carrier. Additional processing steps may then be undertaken to prepare a uniform granulate suitable for formulating into tablets. Sufficient quantities of pharmaceutically necessary tableting excipients may then be admixed with the divalproex granulate, and the resulting mixture may be compressed into tablets.

The divalproex sodium tablets may be coated with an enteric coating to produce delayed-release divalproex sodium tablets. Optionally, a seal coating may also be applied to the tablets before the enteric coating is provided. The enteric coated divalproex sodium tablets may be further overcoated with a film-coating.

In accordance with the invention, the pharmaceutically acceptable carrier may comprise a plurality of inert beads, for example, sugar beads or nonpareil seeds. The neutralized divalproex sodium solution is sprayed onto the inert beads to produce divalproex sodium coated beads, which can then be formulated into solid dosage forms, such as capsules or tablets.

In one embodiment of the invention, the divalproex sodium coated beads may additionally be coated with an enteric coating. In yet another embodiment, a seal coating may be applied to the drug containing beads prior to the application of the enteric coating. After the coatings are applied, the beads may be admixed with sufficient quantities of pharmaceutically necessary tableting excipients. Pharmaceutical tableting excipients include but are not limited to a lubricant, disintegrant, binder, glidant and/or inert diluent. The tablets thus formulated may further be coated with a film-coating.

The invention is further related to a process for preparing divalproex sodium delayed release tablets, wherein the process comprises preparing a neutralized divalproex sodium solution by combining divalproex sodium, having a sodium valproate moiety and a valproic acid moiety, with sodium hydroxide and an aqueous solvent. The base (e.g., sodium hydroxide) is added in sufficient amount to ensure neutralization of the valproic acid moiety of the divalproex sodium. In the neutralized divalproex sodium solution, divalproex sodium does not retain its oligomeric structure or the 1:1 molar ratio of sodium valproate and valproic acid.

The process further comprises spraying the neutralized divalproex sodium solution on a pharmaceutically acceptable carrier and processing the carrier sprayed with the neutralized divalproex sodium solution to obtain divalproex sodium granules. The granules are further processed to obtain divalproex sodium tablet cores, and an enteric coating is applied to the cores to produce divalproex sodium delayed-release tablets. In one example of the invention, a seal coating is applied to the tablet cores prior to the application of the enteric coating. The delayed-release divalproex sodium tablets may also be coated with a film-coating.

In processing the divalproex sodium granules into tablets, as described above, the granules may be admixed with at least one pharmaceutically necessary excipient and compressed into the tablets. Pharmaceutically acceptable excipients include but are not limited to a lubricant, a disintegrant, a binder, a glidant and/or an inert diluent.

In certain embodiments, the invention is directed to an oral solid dosage form comprising a therapeutically effective amount of neutralized divalproex sodium which provides a delayed release of valproate ion when the neutralized divalproex sodium dosage form is orally administered to human patients, said dosage form being bioavailable and providing a therapeutic effect which is bioequivalent to a reference standard which is a delayed release divalproex sodium tablet manufactured by Abbott Laboratories (Depakote®).

In certain embodiments, the invention is directed to an oral solid dosage form comprising a therapeutically effective amount of neutralized divalproex sodium which provides a delayed release of valproate ion when the neutralized divalproex sodium dosage form is orally administered to human patients, said dosage form being bioavailable and providing a therapeutic effect, and said dosage form providing a mean time to maximum plasma concentration (TMAX) of valproic acid at from about 1.8 to about 13.15 hours after oral administration.

In certain embodiments, the invention is directed to an oral solid dosage form comprising a therapeutically effective dose of neutralized divalproex sodium and a pharmaceutically acceptable carrier, said dosage form providing a mean time to maximum plasma concentration (TMAX) of valproic acid at from about 1.8 to about 13.15 hours after oral administration.

The invention is also directed to a method of treating human patients, comprising administering to human patients an effective amounts of the divalproex sodium formulations prepared in accordance with the invention.

The invention is further related to a method of treating complex partial seizures, mania associated with bipolar disorders, and/or migraine headaches in humans comprising orally administering an effective dose of the divalproex sodium formulations prepared in accordance with the invention.

The term “neutralized divalproex sodium,” as used in the present invention, refers to divalproex sodium in which the valproic acid moiety has been neutralized by addition of a strong base, e.g., sodium hydroxide. Neutralized divalproex sodium is not an oligomer. Neutralized divalproex sodium also does not exhibit a 1:1 molar ratio of sodium valproate and valproic acid.

Divalproex sodium tablet prepared using neutralized divalproex sodium solution, therefore, does not contain oligomeric divalproex sodium, nor does it exhibit 1:1 molar ratio of sodium valproate and valproic acid.

### BRIEF DESCRIPTION OF THE DRAWINGS

The following drawings are illustrative of embodiments of the invention and are not meant to limit the scope of the invention as encompassed by the claims.

FIG. 1 is a linear plot comparison of the 500 mg test and 500 mg reference products mean plasma valproic acid concentrations vs time of Example 13.

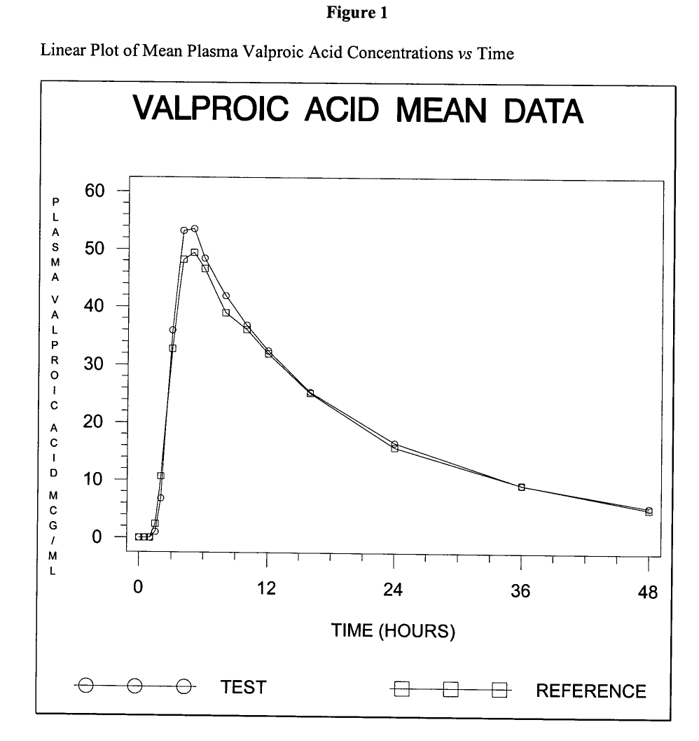

FIG. 2 is semi-logarithmic plot comparison of the 500 mg test and 500 mg reference products mean plasma valproic acid concentrations vs time of Example 13.

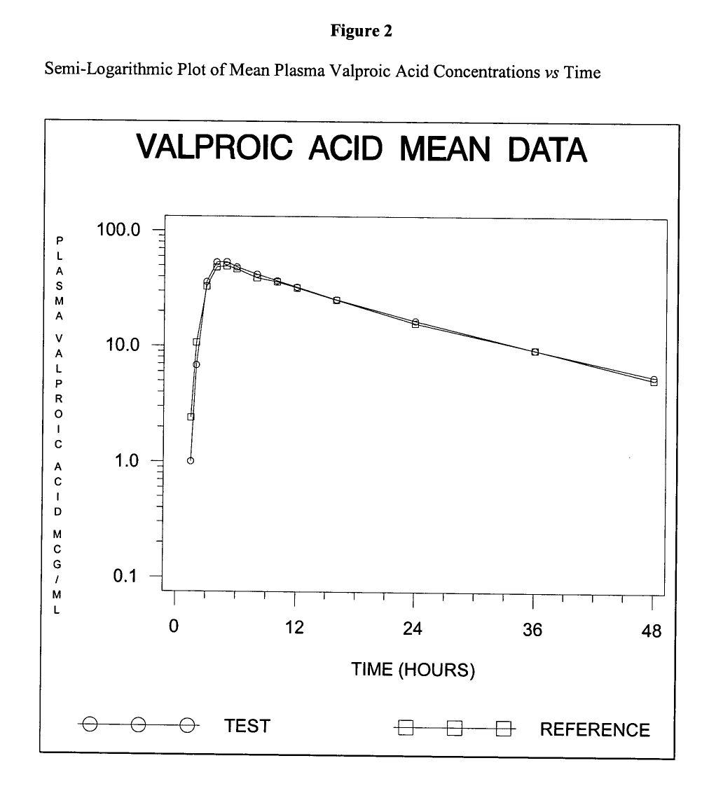

FIG. 3 is a linear plot comparison of the mean plasma valproic acid concentrations vs time for the 500 mg test product in the fasting and fed states, and the 500 mg reference product in the fed state of Example 14.

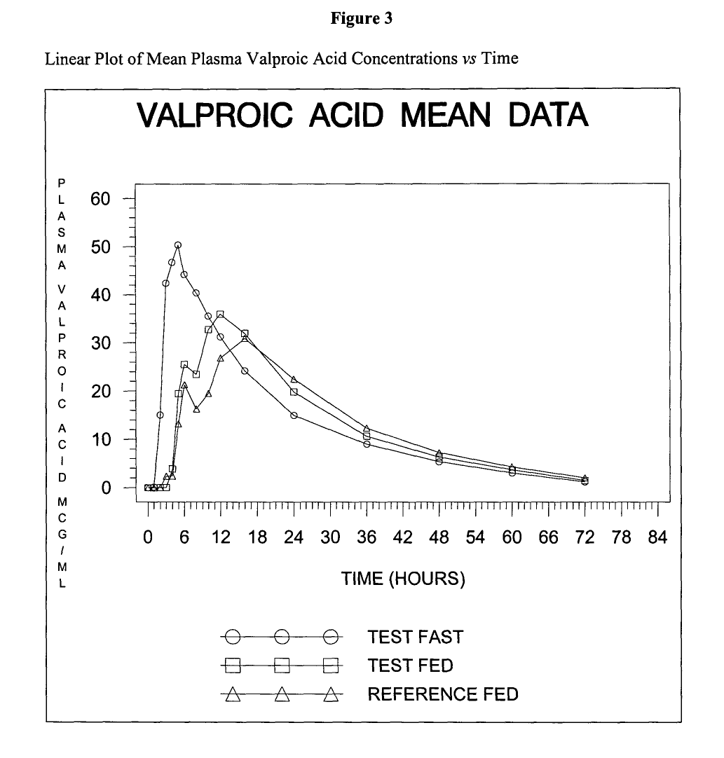

FIG. 4 is semi-logarithmic plot comparison of the mean plasma valproic acid concentrations vs time for the 500 mg test product in the fasting and fed states, and the 500 mg reference product in the fed state of Example 14.

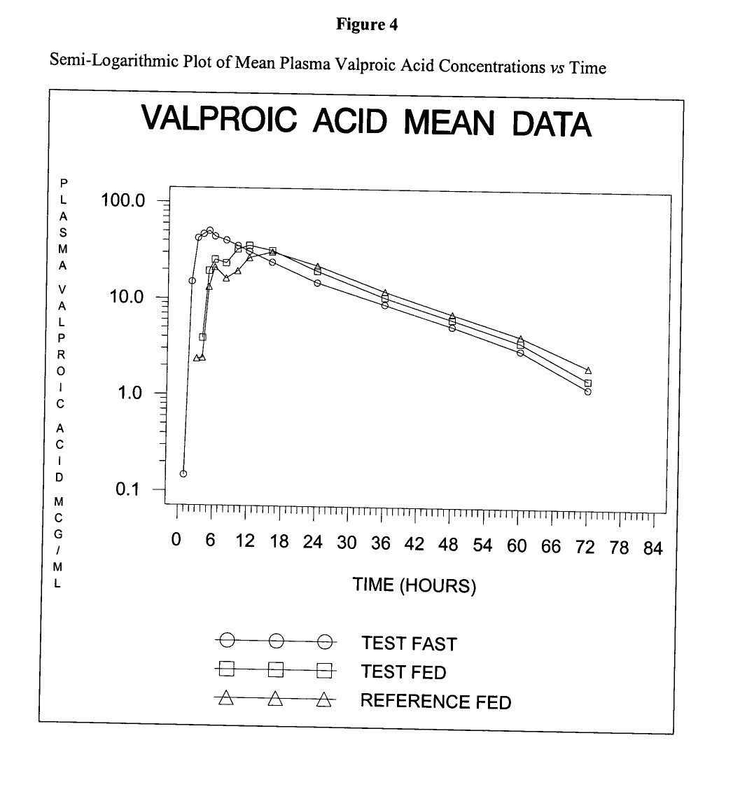

FIG. 5 is a linear plot comparison of the 250 mg test and reference products mean plasma valproic acid concentrations vs time of Example 15.

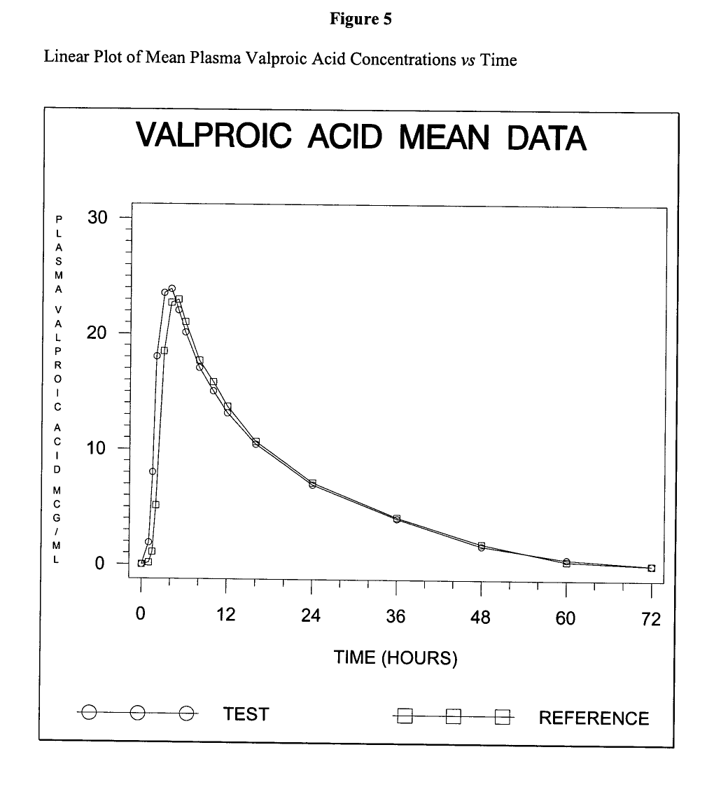

FIG. 6 is semi-logarithmic plot comparison of the 250 mg test and reference products mean plasma valproic acid concentrations vs time of Example 15.

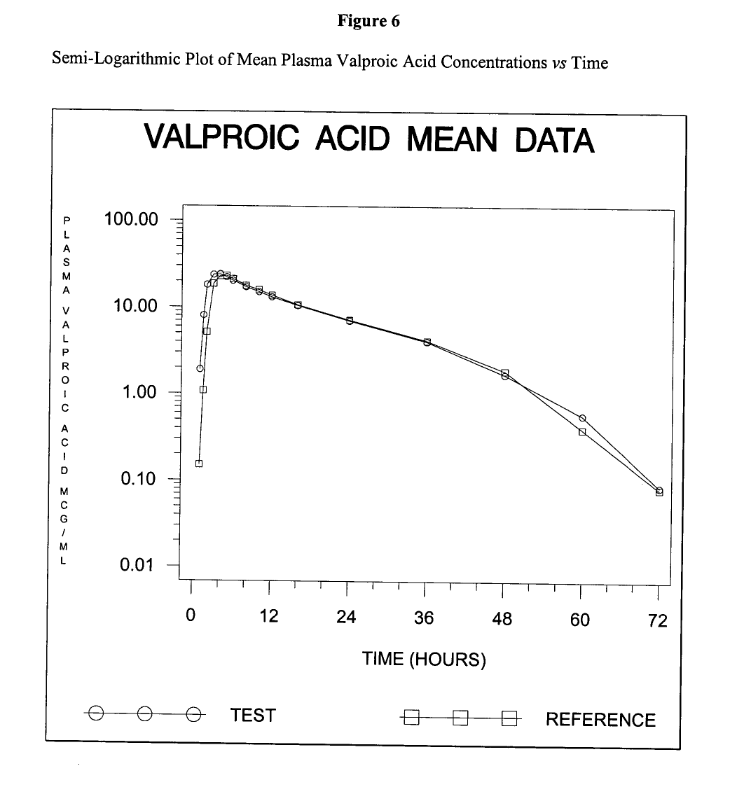

FIG. 7 is a linear plot comparison of the 125 mg test and reference products mean plasma valproic acid concentrations vs time of Example 16.

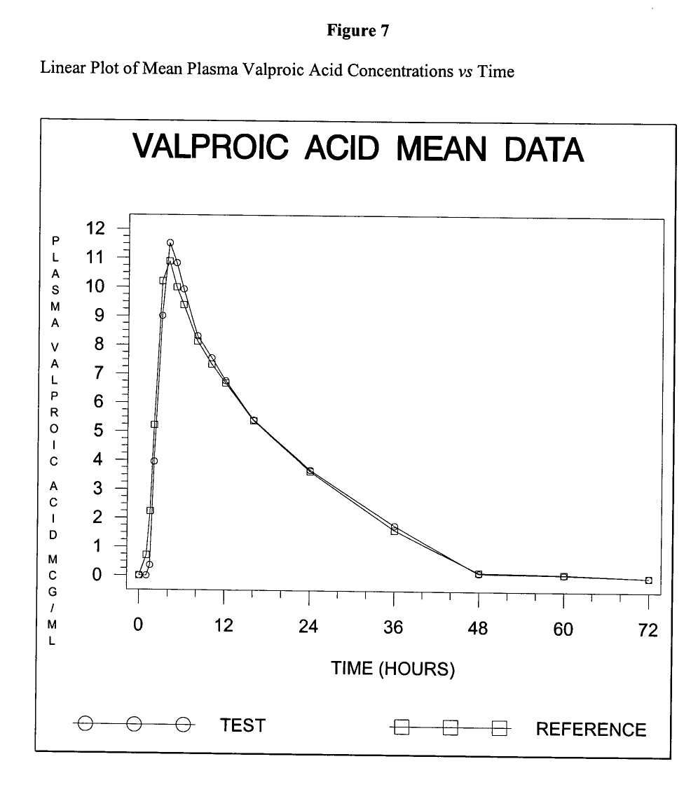

FIG. 8 is semi-logarithmic plot comparison of the 125 mg test and reference products mean plasma valproic acid concentrations vs time of Example 16.

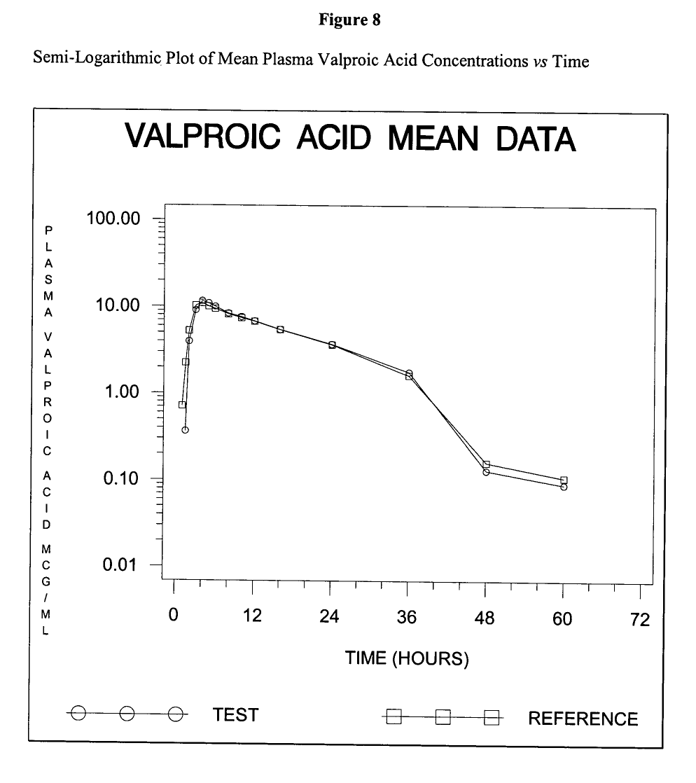

### DETAILED DISCLOSURE OF THE INVENTION

The present invention provides a process for preparing divalproex sodium solid oral dosage forms, where the process comprises preparing a neutralized divalproex sodium solution by combining divalproex sodium with an aqueous solvent and a base, the base added in sufficient quantities to ensure neutralization of the valproic acid moiety of the divalproex sodium. The pH of the neutralized divalproex sodium solution is preferably 10.8±1.0, most preferably ±0.5. In a preferred embodiment, the aqueous solvent is water.

In an embodiment of the invention, the neutralized divalproex sodium solution may be prepared by dissolving divalproex sodium in a basic solution (e.g. sodium hydroxide and water). Additional sodium hydroxide may be added to ensure that the valproic acid moiety of divalproex sodium is neutralized. In a preferred embodiment, additional water is added to the neutralized divalproex sodium solution so that the resulting solution has 20-60%, most preferably 50±3%, valproic acid activity.

In accordance with the present invention, the neutralized divalproex sodium solution is sprayed onto a pharmaceutically acceptable carrier, and the resulting mixture may then be processed to obtain divalproex sodium tablets.

In one embodiment, the pharmaceutically acceptable carrier comprises a plurality of particles of a material that is an inert diluent, and the divalproex sodium solution is sprayed onto the carrier and dried to produce divalproex sodium granules. In another embodiment of the invention, a binder may also be combined with the neutralized divalproex sodium solution and the pharmaceutically acceptable carrier.

In a preferred embodiment of the invention, the neutralized divalproex sodium solution is sprayed onto the pharmaceutically acceptable carrier in a fluid bed processor with a Wurster apparatus. In one embodiment, this process occurs at a product temperature of 42-48° C., with a spray rate of 40-80 ml/min. The divalproex sodium granules may then be dried and then sifted using a mesh screen, e.g., with a 16 mesh screen, to produce divalproex sodium granules.

In a preferred embodiment, the neutralized divalproex sodium solution is diluted, e.g., with isopropyl alcohol before it is sprayed onto the carrier.

The base used in the present invention can be any pharmaceutically acceptable base such as sodium carbonate, sodium bicarbonate, sodium phosphate dibasic, sodium phosphate tribasic, sodium citrate, magnesium hydroxide, magnesium carbonate, calcium carbonate, calcium phosphate, sodium hydroxide and mixtures thereof. A preferred base is sodium hydroxide.

Examples of pharmaceutically acceptable carriers include, but are not limited to, calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose. Further examples of the carrier include hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, and suitable mixtures of the foregoing. A preferred pharmaceutically acceptable carrier is anhydrous lactose.

In certain embodiments, optional pharmaceutical excipients are added to the divalproex sodium granules in the process of formulating the granules into tablets. Such pharmaceutical excipients may include but are not limited to a lubricant, disintegrant, binder, glidant and/or diluent.

Examples of lubricants include magnesium stearate, calcium stearate, oleic acid, caprylic acid, stearic acid, magnesium isovalerate, calcium laurate, magnesium palmitate, behenic acid, glyceryl behenate, glyceryl stearate, sodium stearyl fumarate, potassium stearyl fumarate, and zinc stearate.

Suitable disintegrants include crospovidone, alginates, cellulose and its derivatives, clays, polyvinylpyrrolidone, polysaccharides, such as corn and potato starch, dextrins and sugars. Disintegrants, when used in the formulation, facilitates disintegration when the tablet contacts water in the gastrointestinal tract.

Binders, when added to the formulation, promote granulation and/or promote cohesive compact during the direct compression into tablets. Examples of binders include acacia, cellulose derivatives, gelatin, glucose, polyvinylpyrrolidone, sodium alginate and alginate derivatives, sorbitol, and starch. Binders also include hydrophillic cellulose gums, such as methylcellulose and carboxymethylcellulose, and xanthan gum.

Examples of glidants include but are not limited to corn starch, silica derivatives, and talc.

Examples of inert diluents can include, but are not limited to, calcium phosphate dihydrate, calcium sulfate dihydrate, microcrystalline cellulose, cellulose derivatives, dextrose, lactose, anhydrous lactose, spray-dried lactose, lactose monohydrate, mannitol, starches, sorbitol and sucrose. Further examples of the carrier include hydroxypropylmethylcellulose, hydroxypropylcellulose, methyl cellulose, carboxymethyl cellulose, sodium carboxymethyl cellulose, polyvinylpyrrolidone, polyethyleneglycol, cellulose acetate butyrate, hydroxyethyl cellulose, ethyl cellulose, polyvinyl alcohol, polypropylene, dextrans, dextrins, hydroxypropyl-beta-cyclodextrin, chitosan, copolymers of lactic and glycolic acid, lactic acid polymers, glycolic acid polymers, polyorthoesters, polyanyhydrides, polyvinyl chloride, polyvinyl acetate, ethylene vinyl acetate, lectins, carbopols, silicon elastomers, polyacrylic polymers, maltodextrins, fructose, inositol, trehalose, maltose raffinose, and alpha-, beta-, and gamma-cyclodextrins, and suitable mixtures of the foregoing. A preferred pharmaceutically acceptable carrier is anhydrous lactose.

The tablet cores described above may be coated with an enteric coating to obtain delayed-release divalproex sodium tablets that remain intact in the stomach and release the active ingredient in the intestine. Suitable enteric coating may comprise cellulose acetate phthalate, polyvinyl acetate phthalate, acrylic resins such as Eudragit L.RTM., shellac, cellulose acetate butyrate, hydroxypropyl methylcellulose phthalate or combinations thereof.

Additional materials suitable for use in the enteric coating include phthalates including cellulose acetyl phthalate, cellulose triacetyl phthalate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, and polyvinyl acetate phthalate. The enteric materials are discussed in Remington's Pharmaceutical Sciences, 17th Ed., page 1637 (1985).

The enteric coating may be applied by press coating, molding, spraying, dipping and/or air-suspension or air tumbling procedures. A preferred method of applying the enteric coating is by pan coating, where the enteric coating is applied by spraying the enteric composition onto the tablet cores accompanied by tumbling in a rotating pan. The enteric coating material may be applied to the tablet cores by employing solvents, including an organic , aqueous or a mixture of an organic and aqueous solvent. Examplary solvents suitable in applying the enteric coating include an alcohol, ketone, ester, ether, aliphatic hydrocarbon, halogenated solvents, cycloaliphatic solvents, aromatic, heterocyclic, aqueous solvents, and mixtures thereof. In a preferred embodiment, the enteric coating comprises cellacefate and diethyl phthalate in isopropyl alcohol and acetone. In preferred embodiments, the coating has a thickness from about 6% to about 8% of the final dosage form.

In accordance with the invention, the divalproex sodium tablet cores may further be coated with a seal coating. In a preferred embodiment, the seal coating occurs between the tablet core and the enteric coating. The seal coating may comprise a hydrophilic polymer. Examples include but are not limited to hydroxypropyl cellulose, hydroxypropylmethylcellulose, methoxypropyl cellulose, hydroxypropylisopropylcellulose, hydroxypropylpentylcellulose, hydroxypropylhexylcellulose and any mixtures thereof.

The seal coating, like the enteric coating, may be applied by press coating, molding, spraying, dipping and/or air-suspension or air tumbling procedures. A preferred method of applying the seal coating is by pan coating, where the seal coating is applied by spraying it onto the tablet cores accompanied by tumbling in a rotating pan. The seal coating material may be applied to the tablets as a suspension by employing solvents, e.g., an organic, aqueous, or a mixture of an organic and aqueous solvent. Examplary solvents suitable in applying the seal coating include aqueous-based solutions, an alcohol, ketone, ester, ether, aliphatic hydrocarbon, halogenated solvents, cycloaliphatic solvents, aromatic, heterocyclic, aqueous solvents, and mixtures thereof. In a preferred embodiment, the seal coating comprises hydroxypropyl cellulose and hydroxypropylmethylcellulose, and is delivered as a suspension using ethanol as a solvent.

The divalproex sodium tablets may be overcoated with a pharmaceutically acceptable film coating, e.g., for aesthetic purposes (e.g., including a colorant), for stability purposes (e.g., coated with a moisture barrier), for taste-masking purposes, etc. For example, the tablets may be overcoated with a film coating, preferably containing a pigment and a barrier agent, such as hydroxypropylmethycellulose and/or a polymethylmethacrylate. An example of a suitable material which may be used for such overcoating is hydroxypropylmethylcellulose (e.g., Opadry®, commercially available from Colorcon, West Point, Pa.). In a preferred embodiment, an overcoating is applied to the divalproex sodium tablets that have already been coated with a seal coating and an enteric coating. The overcoat may be applied using a coating pan or a fluidized bed, and may be applied by using a solvent, preferably an aqueous solvent.

The final product is optionally subjected to a polishing step to improve the appearance of the final product and also to facilitate the manipulation of the formulation post manufacture. For example, the slippery nature of the polished dosage form aids in filling printer carrier bars with the formulation and facilitates final packaging of the product. Suitable polishing agents are polyethylene glycols of differing molecular weight or mixtures thereof, talc, surfactants (e.g., Brij types, Myrj types, glycerol mono-stearate and poloxamers), fatty alcohols (e.g., stearyl alcohol, cetyl alcohol, lauryl alcohol and myristyl alcohol) and waxes (e.g., carnauba wax, candelilla wax and white wax). Preferably, polyethylene glycols having molecular weight of 3,000-20,000 are employed.

In certain embodiments of the present invention, the pharmaceutically acceptable carrier onto which the neutralized divalproex sodium solution is sprayed comprises a plurality of inert beads, e.g., sugar beads. The divalproex sodium coated beads thus obtained may be coated with an enteric coating. The beads may also be coated with a seal coating, preferably the seal coating being applied before the enteric coating. The suitable enteric coating and the seal coating materials are set forth above.

The divalproex sodium beads may be formulated into solid oral dosage forms. For example, the beads made be formulated into tablets by admixing them with sufficient quantities of a pharmaceutically necessary tableting excipient and compressing the resulting mixture. The pharmaceutically necessary tableting excipient is selected from the group consisting of a lubricant, a disintegrant, a binder, a glidant, an inert diluent and mixtures thereof. Suitable tableting excipients are set forth above.

In certain preferred embodiments, the present invention provides a process for preparing divalproex sodium delayed-release tablets. The process comprises preparing a neutralized divalproex sodium solution by combining divalproex sodium, having a sodium valproate moiety and a valproic acid moiety, with an aqueous solvent and a base, e.g., sodium hydroxide, the bases being added in sufficient amount to ensure neutralization of the valproic acid moiety of the divalproex sodium. The process further comprises spraying the neutralized divalproex sodium solution onto a pharmaceutically acceptable diluent, processing the resulting mixture to obtain divalproex sodium granules, and processing the granules to obtain tablet cores. An enteric coating is applied to the divalproex sodium tablet cores to produce divalproex sodium delayed-release tablets. Preferably, the delayed-release tablet further comprises a seal coating, applied between the core and the enteric coating. Suitable material for the seal coating and the enteric coating, as well as the procedures for application of these coatings, are set forth above. The tablet thus produced does not contain divalproex sodium that is an oligomeric compound and does not have a 1:1 molar ratio of sodium valproate and valproic acid. Rather, the tablets of the present invention contain divalproex sodium in which the valproic acid moiety has been neutralized.

The pH of the neutralized divalproex sodium solution is preferably about 10.8±0.5, and the neutralized divalproex sodium solution preferably has about 50±3% valproic acid activity. A preferred aqueous solvent for preparation of the neutralized divalproex sodium solution is water.

In a preferred embodiment, the processing of the divalproex sodium granules to obtain tablets comprises drying and then screening the divalproex sodium granules, and admixing the screened divalproex sodium granules with pharmaceutically necessary excipients and compressing the resulting mixture into tablets. The pharmaceutically acceptable excipients are selected from the group consisting of a lubricant, a disintegrant, a binder, a glidant, an inert diluent and mixtures thereof. Examples of suitable excipients are listed above.

In a preferred embodiment, the neutralized divalproex sodium solution is diluted with isopropyl alcohol before it is sprayed onto anhydrous lactose in a fluid bed processor with a Wurster apparatus at product temperature of, e.g., 42-48° C. and a spray rate of, e.g., 40-80 ml/min to form granules. The granules are sized through an appropriate sized screen, e.g., a 16 mesh screen. The sized granules are blended with crospovidone, anhydrous lactose, colloidal silicon dioxide and magnesium stearate and compressed into tablets. The tablets are coated with a seal coating in a coating pan with a suspension of hydroxypropylmethylcellulose, hydroxypropylethylcellulose, hydroxypropyl cellulose and magnesium stearate in ethanol. An enteric coating is then applied, also in a coating pan. The enteric coating comprises cellacefate and diethyl phthalate in isopropyl alcohol and acetone. As an optional final step, the enteric coated tablet is film coated and subjected to a polishing step.

In certain embodiments, the oral solid dosage form of the present invention provides a mean time to maximum plasma concentration (TMAX) of valproic acid at from about 1.8 to about 13.15 hours after oral administration.

In certain embodiments, the oral solid dosage form of the present invention provides a mean time to maximum plasma concentration (TMAX) of valproic acid at from about 2.6 to about 9.1 hours after oral administration.

In certain embodiments, the oral solid dosage form of the present invention provides a mean time to maximum plasma concentration (TMAX) of valproic acid at from about 3.7 to about 9.1 hours after oral administration.

In certain embodiments, the oral solid dosage form of the present invention provides a mean time to maximum plasma concentration (TMAX) of valproic acid at from about 2.7 to about 13.15 hours after oral administration.

In certain embodiments, the oral solid dosage form of the present invention provides a mean time to maximum plasma concentration (TMAX) of valproic acid at from about 2.7 to about 4.8 hours after oral administration in the fasted state.

In certain embodiments, the oral solid dosage form of the present invention provides a mean time to maximum plasma concentration (TMAX) of valproic acid at from about 5 to about 13.15 hours after oral administration in the fed state.

In certain embodiments, the oral solid dosage form of the present invention provides a mean time to maximum plasma concentration (TMAX) of valproic acid at from about 1.8 to about 3.5 hours after oral administration.

In certain embodiments, the oral solid dosage form of the present invention provides a mean time to maximum plasma concentration (TMAX) of valproic acid at from about 2.4 to about 4 hours after oral administration.

In certain embodiments, the oral solid dosage form of the present invention provides a mean maximum plasma concentration (CMAX) of valproic acid of from about 38 to about 67 mcg/ml, based on oral administration of a 500 mg delayed release dose of neutralized divalproex sodium.

In certain embodiments, the oral solid dosage form of the present invention provides a mean maximum plasma concentration (CMAX) of valproic acid of from about 49 to about 59 mcg/ml, based on oral administration of a 500 mg delayed release dose of neutralized divalproex sodium.

In certain embodiments, the oral solid dosage form of the present invention provides a mean maximum plasma concentration (CMAX) of valproic acid of from about 46 to about 67 mcg/ml, based on oral administration in the fasted state of a 500 mg delayed release dose of neutralized divalproex sodium.

In certain embodiments, the oral solid dosage form of the present invention provides a mean maximum plasma concentration (CMAX) of valproic acid of from about 38 to about 59 mcg/ml, based on oral administration in the fed state of a 500 mg delayed release dose of neutralized divalproex sodium.

In certain embodiments, the oral solid dosage form of the present invention provides a mean maximum plasma concentration (CMAX) of valproic acid of from about 23 to about 33 mcg/ml, based on oral administration of a 250 mg delayed release dose of neutralized divalproex sodium.

In certain embodiments, the oral solid dosage form of the present invention provides a mean maximum plasma concentration (CMAX) of valproic acid of from about 11 to about 15 mcg/ml, based on oral administration of a 125 mg delayed release dose of neutralized divalproex sodium.

In certain embodiments, the oral solid dosage form of the present invention provides a mean AUC from time zero to last quantifiable concentration (AUCTLQC) of valproic acid of from about 763 to about 1154 mcg·hr/ml, based on oral administration of a 500 mg delayed release dose of neutralized divalproex sodium.

In certain embodiments, the oral solid dosage form of the present invention provides a mean AUC from time zero to last quantifiable concentration (AUCTLQC) of valproic acid of from about 925 to about 976 mcg·hr/ml, based on oral administration of a 500 mg delayed release dose of neutralized divalproex sodium.

In certain embodiments, the oral solid dosage form of the present invention provides a mean AUC from time zero to last quantifiable concentration (AUCTLQC) of valproic acid of from about 771 to about 1154 mcg·hr/ml, based on oral administration in the fasted state of a 500 mg delayed release dose of neutralized divalproex sodium.

In certain embodiments, the oral solid dosage form of the present invention provides a mean AUC from time zero to last quantifiable concentration (AUCTLQC) of valproic acid of from about 763 to about 1087 mcg·hr/ml, based on oral administration in the fed state of a 500 mg delayed release dose of neutralized divalproex sodium.

In certain embodiments, the oral solid dosage form of the present invention provides a mean AUC from time zero to last quantifiable concentration (AUCTLQC) of valproic acid of from about 323 to about 509 mcg·hr/ml, based on oral administration of a 250 mg delayed release dose of neutralized divalproex sodium.

In certain embodiments, the oral solid dosage form of the present invention provides a mean AUC from time zero to last quantifiable concentration (AUCTLQC) of valproic acid of from about 127 to about 231 mcg·hr/ml, based on oral administration of a 125 mg delayed release dose of neutralized divalproex sodium.

In certain embodiments, the oral solid dosage form of the present invention provides a mean AUC from time zero to infinity (AUCINF) of valproic acid of 1062±227 mcg·hr/ml based on oral administration in the fasted state of a 500 mg delayed release dose of neutralized divalproex sodium.

In certain embodiments, the oral solid dosage form of the present invention provides a mean AUC from time zero to infinity (AUCINF) of valproic acid of about 988 to about 1062 mcg·hr/ml based on oral administration in the fasted state of a 500 mg delayed release dose of neutralized divalproex sodium.

In certain embodiments, the oral solid dosage form of the present invention provides a mean AUC from time zero to infinity (AUCINF) of valproic acid of 1038±192 mcg·hr/ml based on oral administration in the fasted state of a 500 mg delayed release dose of neutralized divalproex sodium.

In certain embodiments, the oral solid dosage form of the present invention provides a mean AUC from time zero to infinity (AUCINF) of valproic acid of 988±173 mcg·hr/ml based on oral administration in the fed state of a 500 mg delayed release dose of neutralized divalproex sodium.

In certain embodiments, the oral solid dosage form of the present invention provides a mean AUC from time zero to infinity (AUCINF) of valproic acid of 471±100 mcg·hr/ml based on oral administration of a 250 mg delayed release dose of neutralized divalproex sodium.

In certain embodiments, the oral solid dosage form of the present invention provides a mean AUC from time zero to infinity (AUCINF) of valproic acid of 235±63 mcg·hr/ml based on oral administration of a 125 mg delayed release dose of neutralized divalproex sodium.

In certain embodiments, the oral solid dosage form of the present invention provides a mean AUC to last quantifiable concentration (AUCTLQC) of valproic acid which is from about 80% to about 125% of the AUC to last quantifiable concentration (AUCTLQC) of valproic acid provided by oral administration of a reference standard over the same time interval.

In certain embodiments, the oral solid dosage form of the present invention provides a mean AUC to last quantifiable concentration (AUCTLQC) of valproic acid which is from about 80% to about 125% of the AUC to last quantifiable concentration (AUCTLQC) of valproic acid provided by oral administration of a reference standard over the same time interval, and wherein the dosage form and the reference standard are orally administered in a fasted state.

In certain embodiments, the oral solid dosage form of the present invention provides a mean AUC to last quantifiable concentration (AUCTLQC) of valproic acid which is from about 80% to about 125% of the AUC to last quantifiable concentration (AUCTLQC) of valproic acid provided by oral administration of a reference standard over the same time interval, and wherein the dosage form and the reference standard are orally administered in a fed state.

In certain embodiments, the oral solid dosage form of the present invention provides provides a mean half-life (THALF) of valproic acid of about 10.9 to about 21.3 hours.

### DETAILED DESCRIPTION OF THE PREFERRED EMBODIMENTS

The following example illustrate various aspects of the present invention. It is not to be construed to limit the claims in any manner whatsoever.

### EXAMPLE 1

### Divalproex Sodium Delayed Release Tablets

1. Preparation of Neutralized Divalproex Sodium Solution

Neutralized divalproex sodium solution is prepared by dissolving 260 kg of divalproex sodium in about 189.49 kg purified water with 33.51 kg of sodium hydroxide. The solution is adjusted to pH 10.8±0.3 with 20% sodium hydroxide solution and adjusted to 483 kg with additional purified water to yield divalproex sodium solution with 50±3% valproic acid activity.

2. Preparation Divalproex Sodium Granules

11.52 kg of the neutralized divalproex sodium solution is diluted with 14.57 kg of isopropyl alcohol. The diluted solution is then sprayed onto 5.15 kg anhydrous lactose in a fluid bed processor with a Wurster apparatus at product temperature of 42-48° C. and spray rate of 40-80 ml/min to form divalproex sodium granules. The granules are sized through a sifter equipped with 16 mesh screen.

3. Blending and Tableting

The sifted divalproex sodium granules, 102.51 kg, are blended with 3.987 kg crospovidone, 5.695 kg anhydrous lactose, 0.57 kg colloidal silicon dioxide and 1.139 magnesium stearate to yield divalproex sodium blend. The divalproex sodium blend is then compressed to yield divalproex sodium tablets having a weight of 871 to 983 mg, with 500 mg valproic acid activity.

4. Seal Coating and Enteric Coating

The divalproex sodium tablet cores, 108.3 kg, are seal coated in a coating pan with a suspension of 1.34 kg hydroxypropylmethylcellulose, 1.34 kg hydroxypropylcellulose and 0.67 kg magnesium stearate in 30.15 kg ethanol. The seal coated tablets, 110.71 kg, are coated in a coating pan with a solution of 7.181 kg cellacefate (CAP) and 1.795 kg diethyl phthalate in 31.42 kg ispropyl alcohol and 31.42 kg acetone to yield enteric coated, delayed-release divalproex sodium tablets.

5. Color Coating and Polishing

The enteric coated tablets, 118.51 kg, are color coated with a solution of 3.291 kg Opadry Blue and 0.037 kg Vanillin in 29.62 kg water. The color coated tablets are then polished by sprinkling 0.037 kg candelilla wax powder onto the tablets while the pan is rotating to yield color-coated divalproex sodium delayed-release tablets, with 500 mg valproic acid activity.

The example provided above is not meant to be exclusive. Many other variations of the present invention would be obvious to those skilled in the art, and are contemplated to be within the scope of the appended claims. For example, it will be recognized by those skilled in the art that a wide variety of pharmaceutically acceptable excipients may be utilized for their intended purpose in the process for preparing divalproex sodium tablets as described herein.

The percent (%) ingredients for tablets prepared in accordance with Example 1 are listed in Table 1 below:

TABLE 1

| Ingredient | Percent (%) of composition |
|---|---|
| Divalproex Sodium Granules | |
| Divalproex Sodium Solution | 69.11 |
| Anhydrous Lactose | 30.89 |
| Subtotal | 100.00 |
| Divalproex Sodium Blend | |
| Divalproex Sodium Granules | 90.00 |
| Crosspovidone, NF | 3.50 |
| Anhydrous Lactose, NF | 5.00 |
| Colloidal Silicon Dioxide, NF | 0.50 |
| Magnesium Stearate, NF | 1.00 |
| Subtotal | 100.00 |
| (This total blend was compressed into tablets | |
| as in Example 1) | |
| Seal Coating | |
| Divalproex Sodium tablets (compressed | 97.00 |
| blend) | |
| Hydroxypropylmethylcellulose, USP | 1.20 |
| (Methocel E5 Premium) | |
| Hydroxypropyl Cellulose, USP (Klucel, | 1.20 |
| EF) | |
| Magnesium Stearate, NF | 0.60 |
| Subtotal | 100.00 |
| Enteric Coating | |
| Divalproex Sodium(Seal Coated) Tablets | 91.00 |
| Cellacefate, NF | 7.20 |
| Diethyl Pthalate | 1.80 |
| Subtotal | 100.00 |
| Color Coat and Polishing | |
| Divalproex Sodium (Enteric Coated) | 97.24 |
| Tablets | |
| Opadry Blue | 2.70 |
| Vanillin, NF | 0.03 |
| Candilla Wax | 0.03 |
| Total | 100.00 |

### EXAMPLES 2-10

In Example 2, divalproex delayed release tablets were prepared in accordance with Example 1, with an equivalent amount of sodium carbonate substituted for the sodium hydroxide.

In Example 3, divalproex delayed release tablets were prepared in accordance with Example 1, with an equivalent amount of sodium bicarbonate substituted for the sodium hydroxide.

In Example 4, divalproex delayed release tablets were prepared in accordance with Example 1, with an equivalent amount of sodium phosphate dibasic substituted for the sodium hydroxide.

In Example 5, divalproex delayed release tablets were prepared in accordance with Example 1, with an equivalent amount of sodium phosphate tribasic substituted for the sodium hydroxide.

In Example 6, divalproex delayed release tablets were prepared in accordance with Example 1, with an equivalent amount of sodium citrate substituted for the sodium hydroxide.

In Example 7, divalproex delayed release tablets were prepared in accordance with Example 1, with an equivalent amount of magnesium hydroxide substituted for the sodium hydroxide.

In Example 8, divalproex delayed release tablets were prepared in accordance with Example 1, with an equivalent amount of magnesium carbonate substituted for the sodium hydroxide.

In Example 9, divalproex delayed release tablets were prepared in accordance with Example 1, with an equivalent amount of calcium carbonate substituted for the sodium hydroxide.

In Example 10, divalproex delayed release tablets were prepared in accordance with Example 1, with an equivalent amount of calcium phosphate substituted for the sodium hydroxide.

### EXAMPLE 11

In Example 11, 250 mg divalproex sodium tablets were prepared in accordance with the process of Example 1 and having the following ingredients in the respective percentages listed in the Table 2 below:

TABLE 2

| Ingredient | Percent (%) of composition |
|---|---|
| Divalproex Sodium Granules | |
| Divalproex Sodium Solution | 69.11 |
| Anhydrous Lactose | 30.89 |
| Subtotal | 100.00 |
| Divalproex Sodium Blend | |
| Divalproex Sodium Granules | 90.00 |
| Crosspovidone, NF | 3.50 |
| Anhydrous Lactose, NF | 5.00 |
| Colloidal Silicon Dioxide, NF | 0.50 |
| Magnesium Stearate, NF | 1.00 |
| Subtotal | 100.00 |
| (This total blend was compressed into tablets | |
| as in Example 1) | |
| Seal Coating | |
| Divalproex Sodium tablets (compressed | 97.00 |
| blend) | |
| Hydroxypropylmethylcellulose, USP | 1.20 |
| (Methocel E5 Premium) | |
| Hydroxypropyl Cellulose, USP (Klucel, | 1.20 |
| EF) | |
| Magnesium Stearate, NF | 0.60 |
| Subtotal | 100.00 |
| Enteric Coating | |
| Divalproex Sodium(Seal Coated) Tablets | 91.00 |
| Cellacefate, NF | 7.20 |
| Diethyl Pthalate | 1.80 |
| Subtotal | 100.00 |
| Color Coat and Polishing | |
| Divalproex Sodium (Enteric Coated) | 97.17 |
| Tablets | |
| Opadry Blue | 2.75 |
| Vanillin, NF | 0.04 |
| Candilla Wax | 0.04 |
| Total | 100.00 |

### EXAMPLE 12

In Example 12, 125 mg divalproex sodium tablets were prepared in accordance with the process of Example 1 and having the following ingredients in the respective percentages listed in the Table 3 below:

TABLE 3

| Ingredient | Percent (%) of composition |
|---|---|
| Divalproex Sodium Granules | |
| Divalproex Sodium Solution | 69.11 |
| Anhydrous Lactose | 30.89 |
| Subtotal | 100.00 |
| Divalproex Sodium Blend | |
| Divalproex Sodium Granules | 90.00 |
| Crosspovidone, NF | 3.50 |
| Anhydrous Lactose, NF | 5.00 |
| Colloidal Silicon Dioxide, NF | 0.50 |
| Magnesium Stearate, NF | 1.00 |
| Subtotal | 100.00 |
| (This total blend was compressed into tablets | |
| as in Example 1) | |
| Seal Coating | |
| Divalproex Sodium tablets (compressed | 97.00 |
| blend) | |
| Hydroxypropylmethylcellulose, USP | 1.20 |
| (Methocel E5 Premium) | |
| Hydroxypropyl Cellulose, USP (Klucel, | 1.20 |
| EF) | |
| Magnesium Stearate, NF | 0.60 |
| Subtotal | 100.00 |
| Enteric Coating | |
| Divalproex Sodium(Seal Coated) Tablets | 87.00 |
| Cellacefate, NF | 10.40 |
| Diethyl Pthalate | 2.60 |
| Subtotal | 100.00 |
| Color Coat | |
| Divalproex Sodium (Enteric Coated) | 96.97 |
| Tablets | |
| Opadry Blue | 3.00 |
| Vanillin, NF | 0.03 |
| Candilla Wax | — |
| Subtotal | 100.00 |
| Wax polish | |
| Divalproex Sodium Color Coated Tablets | 99.97 |
| Candilla Wax | 0.03 |
| Total | 100.00 |

### EXAMPLE 13

In Example 13, a randomized, single-dose, open label, two-way crossover study design was used to compare the oral bioavailability of a 500 mg delayed release test divalproex sodium formulation prepared according to Example 1, to an equivalent oral dose of a commercially available 500 mg delayed release divalproex sodium (Depakote®, Abbott Laboratories, Lot No. 45-404-AA-21, exp. date Oct. 1, 2001), in a test population of 30 healthy adult males under fasting conditions. Sampling times were at hour 0 (within 60 minutes prior to dose), and at post-dosing hours 0.5, 1, 1.5, 2, 3, 4, 5, 6, 8, 10, 12, 16, 24, 36, and 48. A total of 16 samples per subject were collected in each study period. All subjects consumed 240 ml (8 fl. ozs.) of room temperature tap water simultaneously, at 1.5 hours prior to each dosing, and 120 ml (4 fl. ozs.) of room temperature tap water at post-dose hours 1, 2, and 3, after each dose.

The following pharmacokinetic parameters were determined from the plasma concentration data.

The area under the plasma concentration versus time curve [AUCTLQC] was calculated using the linear trapezoidal rule from the zero time point to the last quantifiable concentration.

The area under the plasma concentration versus time curve from zero to infinity [AUCINF] was calculated by adding Cₜ/KELM to AUCTLQC where Cₜ is the last quantifiable concentration and KELM is the elimination rate constant.

The maximum observed plasma concentration [CMAX] was obtained by inspection. The time to maximum plasma concentration [TMAX] was obtained by inspection.

The terminal elimination rate constant [KELM] was obtained from the slope of the line, fitted by linear least squares regression, through the terminal points of the log(base e) of the concentration versus time plot for these points.

The half-life [THALF] was calculated by the equation THALF=0.693/KELM.

The lag time was obtained by inspection, and is the time to the first quantifiable concentration. Throughout this report the lag time will be designated as TLAG.

The elimination of valproic acid from the plasma appeared to be polyphasic for most of the subjects. The elimination rate constants were estimated from the plasma valproic acid data for all subjects using the plasma concentrations of the elimination phase as determined from the plasma drug concentration vs time plots (log scale) for the individual subjects.

The number of values used in the mean calculations is designated as “N”.

The coefficient of variation is designated as “CV”.

All concentrations are reported as mcg/ml.

The mean plasma valproic acid concentration (mcg/ml) by time point values for the 500 mg test product are listed in Table 4 below:

TABLE 4

| Variable* | Time | N | Mean | Std Dev | CV |
|---|---|---|---|---|---|
| C1 | 0.00 HR | 30 | 0.000 | 0.000 | . |
| C2 | 0.50 HR | 30 | 0.000 | 0.000 | . |
| C3 | 1.00 HR | 30 | 0.000 | 0.000 | . |
| C4 | 1.50 HR | 30 | 1.007 | 4.223 | 419.487 |
| C5 | 2.00 HR | 30 | 6.821 | 16.008 | 234.682 |
| C6 | 3.00 HR | 30 | 35.939 | 25.078 | 69.780 |
| C7 | 4.00 HR | 30 | 53.220 | 13.486 | 25.341 |
| C8 | 5.00 HR | 30 | 53.530 | 8.153 | 15.230 |
| C9 | 6.00 HR | 30 | 48.433 | 6.459 | 13.337 |
| C10 | 8.00 HR | 30 | 41.917 | 6.787 | 16.192 |
| C11 | 10.0 HR | 30 | 36.853 | 6.640 | 18.018 |
| C12 | 12.0 HR | 30 | 32.477 | 6.333 | 19.499 |
| C13 | 16.0 HR | 30 | 25.370 | 5.646 | 22.254 |
| C14 | 24.0 HR | 30 | 16.590 | 4.008 | 24.161 |
| C15 | 36.0 HR | 30 | 9.310 | 2.860 | 30.719 |
| C16 | 48.0 HR | 30 | 5.535 | 2.289 | 41.353 |

*C1-C16 designate the mean concentrations at the corresponding time points.

The mean plasma valproic acid concentration (mcg/ml) by time point values for the 500 mg reference product are listed in Table 5 below:

TABLE 5

| Variable | Time | N | Mean | Std Dev | CV |
|---|---|---|---|---|---|
| C1 | 0.00 HR | 30 | 0.000 | 0.000 | . |
| C2 | 0.50 HR | 30 | 0.000 | 0.000 | . |
| C3 | 1.00 HR | 30 | 0.000 | 0.000 | . |
| C4 | 1.50 HR | 30 | 2.411 | 6.805 | 282.222 |
| C5 | 2.00 HR | 30 | 10.666 | 17.813 | 167.010 |
| C6 | 3.00 HR | 30 | 32.770 | 27.142 | 82.826 |
| C7 | 4.00 HR | 30 | 48.193 | 14.491 | 30.069 |
| C8 | 5.00 HR | 30 | 49.414 | 10.230 | 20.703 |
| C9 | 6.00 HR | 30 | 46.633 | 10.055 | 21.562 |
| C10 | 8.00 HR | 30 | 38.970 | 5.182 | 13.297 |
| C11 | 10.0 HR | 30 | 36.117 | 5.734 | 15.875 |
| C12 | 12.0 HR | 30 | 31.997 | 5.484 | 17.139 |
| C13 | 16.0 HR | 30 | 25.177 | 5.263 | 20.904 |
| C14 | 24.0 HR | 30 | 15.816 | 3.859 | 24.397 |
| C15 | 36.0 HR | 30 | 9.348 | 3.090 | 33.057 |
| C16 | 48.0 HR | 30 | 5.219 | 2.354 | 45.108 |

The mean (arithmetic) pharmacokinetic values for the 500 mg test product are listed in Table 6 below:

TABLE 6

| Variable | N | Mean | Std Dev | CV |
|---|---|---|---|---|
| AUCTLQC | 30 | 939.035 | 167.478 | 17.835 |
| AUCINF | 30 | 1062.032 | 227.412 | 21.413 |
| CMAX | 30 | 58.757 | 8.154 | 13.877 |
| TMAX | 30 | 3.867 | 0.819 | 21.189 |
| KELM | 30 | 0.051 | 0.010 | 19.526 |
| THALF | 30 | 14.289 | 3.188 | 22.310 |
| TLAG | 30 | 2.817 | 0.886 | 31.440 |
| LAUCTLQC* | 30 | 6.830 | 0.176 | 2.573 |
| LAUCINF* | 30 | 6.947 | 0.208 | 2.989 |
| LCMAX* | 30 | 4.064 | 0.135 | 3.318 |

*Least squares mean log data.

The mean (arithmetic) pharmacokinetic values for the 500 mg reference product are listed in Table 7 below:

TABLE 7

| Variable | N | Mean | Std Dev | CV |
|---|---|---|---|---|
| AUCTLQC | 30 | 908.605 | 146.360 | 16.108 |
| AUCINF | 30 | 1023.595 | 221.609 | 21.650 |
| CMAX | 30 | 57.720 | 9.330 | 16.164 |
| TMAX | 30 | 4.267 | 1.760 | 41.258 |
| KELM | 30 | 0.052 | 0.010 | 19.794 |
| THALF | 30 | 13.959 | 3.338 | 23.912 |
| TLAG | 30 | 2.817 | 0.987 | 35.036 |
| LAUCTLQC | 30 | 6.800 | 0.155 | 2.285 |
| LAUCINF | 30 | 6.911 | 0.197 | 2.844 |
| LCMAX | 30 | 4.044 | 0.156 | 3.869 |

The results of this study indicate that the test product is bioequivalent to the reference product.

### EXAMPLE 14

In Example 14, a randomized, single-dose, open label, three-way crossover study design was used to evaluate the relative bioavailability of a 500 mg test divalproex sodium formulation prepared according to Example 11, to an equivalent dose of the commercially available 500 mg delayed release divalproex sodium (Depakote®, Abbott Laboratories, Lot No. 45-404-AA-21, exp. date Oct. 1, 2001), in a test population of 24 healthy adult males under fasted and fed conditions. Sampling times were at hour 0 (within 60 minutes prior to dose), and at post-dosing hours 1, 2, 3, 4, 5, 6, 8, 10, 12, 16, 24, 36, 48, 60, 72 and 96. A total of 17 samples per subject were collected in each study period. All subjects consumed 240 ml (8 fl. ozs.) of room temperature tap water simultaneously, at 1.5 hours prior to each dosing, and 120 ml (4 fl. ozs.) of room temperature tap water at post-dose hours 1, 2, and 3, after each dose.

Subjects assigned to the fed groups were given a standardized meal starting 15 minutes before their assigned dose time.

Pharmacokinetic parameters were determined according to the procedure in Example 13. All concentrations are reported as mcg/ml.

The mean plasma valproic acid fasting concentration (mcg/ml) by time point values for the 500 mg test product are listed in Table 8 below:

TABLE 8

| Variable | Time | N | Mean | Std Dev | CV |
|---|---|---|---|---|---|
| C1 | 0.00 HR | 22 | 0.000 | 0.000 | . |
| C2 | 1.00 HR | 22 | 0.147 | 0.691 | 469.042 |
| C3 | 2.00 HR | 22 | 15.025 | 19.703 | 131.141 |
| C4 | 3.00 HR | 22 | 42.406 | 20.917 | 49.325 |
| C5 | 4.00 HR | 22 | 46.773 | 16.952 | 36.243 |
| C6 | 5.00 HR | 22 | 50.391 | 11.675 | 23.169 |
| C7 | 6.00 HR | 22 | 44.218 | 5.725 | 12.947 |
| C8 | 8.00 HR | 22 | 40.391 | 10.286 | 25.465 |
| C9 | 10.0 HR | 22 | 35.564 | 10.183 | 28.633 |
| C10 | 12.0 HR | 22 | 31.200 | 8.050 | 25.802 |
| C11 | 16.0 HR | 22 | 24.168 | 5.320 | 22.012 |
| C12 | 24.0 HR | 22 | 14.989 | 3.597 | 23.996 |
| C13 | 36.0 HR | 20 | 9.006 | 2.391 | 26.547 |
| C14 | 48.0 HR | 21 | 5.377 | 2.311 | 42.971 |
| C15 | 60.0 HR | 21 | 3.060 | 1.517 | 49.578 |
| C16 | 72.0 HR | 21 | 1.246 | 1.545 | 124.038 |
| C17 | 96.0 HR | 21 | 0.137 | 0.626 | 458.258 |

The mean plasma valproic acid fed valproic acid fed concentration (mcg/ml) by time point values for the 500 mg test product are listed in Table 9 below:

TABLE 9

| Variable | Time | N | Mean | Std Dev | CV |
|---|---|---|---|---|---|
| C1 | 0.00 HR | 22 | 0.000 | 0.000 | . |
| C2 | 1.00 HR | 22 | 0.000 | 0.000 | . |
| C3 | 2.00 HR | 22 | 0.000 | 0.000 | . |
| C4 | 3.00 HR | 22 | 0.000 | 0.000 | . |
| C5 | 4.00 HR | 22 | 3.891 | 13.527 | 347.659 |
| C6 | 5.00 HR | 22 | 19.496 | 23.577 | 120.932 |
| C7 | 6.00 HR | 22 | 25.509 | 26.927 | 105.558 |
| C8 | 8.00 HR | 22 | 23.448 | 21.423 | 91.365 |
| C9 | 10.0 HR | 22 | 32.722 | 13.825 | 42.251 |
| C10 | 12.0 HR | 22 | 35.936 | 8.575 | 23.861 |
| C11 | 16.0 HR | 22 | 31.909 | 8.215 | 25.745 |
| C12 | 24.0 HR | 22 | 19.818 | 4.940 | 24.928 |
| C13 | 36.0 HR | 22 | 10.645 | 2.642 | 24.822 |
| C14 | 48.0 HR | 22 | 6.353 | 1.909 | 30.052 |
| C15 | 60.0 HR | 22 | 3.695 | 1.385 | 37.492 |
| C16 | 72.0 HR | 22 | 1.529 | 1.486 | 97.226 |
| C17 | 96.0 HR | 21 | 0.116 | 0.532 | 458.258 |

The mean plasma valproic acid fed concentration (mcg/ml) by time point values for the 500 mg reference product are listed in Table 10.

TABLE 10

| Variable | Time | N | Mean | Std Dev | CV |
|---|---|---|---|---|---|
| C1 | 0.00 HR | 22 | 0.000 | 0.000 | . |
| C2 | 1.00 HR | 22 | 0.000 | 0.000 | . |
| C3 | 2.00 HR | 22 | 0.000 | 0.000 | . |
| C4 | 3.00 HR | 22 | 2.364 | 11.086 | 469.042 |
| C5 | 4.00 HR | 22 | 2.418 | 11.342 | 469.042 |
| C6 | 5.00 HR | 22 | 13.212 | 20.913 | 158.291 |
| C7 | 6.00 HR | 22 | 21.332 | 27.241 | 127.703 |
| C8 | 8.00 HR | 22 | 16.278 | 20.201 | 124.101 |
| C9 | 10.0 HR | 22 | 19.528 | 19.205 | 98.344 |
| C10 | 12.0 HR | 22 | 26.820 | 16.635 | 62.026 |
| C11 | 16.0 HR | 22 | 30.877 | 10.841 | 35.110 |
| C12 | 24.0 HR | 22 | 22.476 | 7.020 | 31.233 |
| C13 | 36.0 HR | 22 | 12.324 | 3.672 | 29.800 |
| C14 | 48.0 HR | 22 | 7.265 | 2.749 | 37.837 |
| C15 | 60.0 HR | 22 | 4.285 | 1.654 | 38.605 |
| C16 | 72.0 HR | 22 | 2.081 | 1.622 | 77.936 |
| C17 | 96.0 HR | 22 | 0.194 | 0.628 | 324.332 |

The mean (arithmetic) pharmacokinetic values for the 500 mg test product in the fasting and fed state and the 500 mg reference product in the fed state are listed in Table 11 below:

TABLE 11

| | | | | |
|---|---|---|---|---|
| Variable | N | Mean | Std Dev | CV |

500 mg test product fasting

| | | | | |
|---|---|---|---|---|
| AUCTLQC | 21 | 975.674 | 177.522 | 18.195 |
| AUCINF | 21 | 1038.910 | 192.703 | 18.549 |
| CMAX | 22 | 56.159 | 9.937 | 17.695 |
| TMAX | 22 | 3.727 | 1.032 | 27.687 |
| KELM | 22 | 0.048 | 0.018 | 37.480 |
| THALF | 22 | 16.102 | 5.198 | 32.280 |
| TLAG | 22 | 2.591 | 0.959 | 37.019 |
| LAUCTLQC | 21 | 6.868 | 0.173 | 2.521 |
| LAUCINF | 21 | 6.931 | 0.174 | 2.509 |
| LCMAX | 22 | 4.015 | 0.164 | 4.082 |

500 mg test product fed

| | | | | |
|---|---|---|---|---|
| AUCTLQC | 22 | 925.079 | 161.598 | 17.469 |
| AUCINF | 22 | 988.230 | 173.785 | 17.585 |
| CMAX | 22 | 48.718 | 9.948 | 20.420 |
| TMAX | 22 | 9.091 | 4.058 | 44.638 |
| KELM | 22 | 0.047 | 0.013 | 27.000 |
| THALF | 22 | 15.926 | 4.820 | 30.267 |
| TLAG | 22 | 6.955 | 2.319 | 33.348 |
| LAUCTLQC | 22 | 6.816 | 0.168 | 2.463 |
| LAUCINF | 22 | 6.882 | 0.169 | 2.455 |
| LCMAX | 22 | 3.866 | 0.204 | 5.287 |

500 mg reference product fed

| | | | | |
|---|---|---|---|---|
| AUCTLQC | 22 | 913.104 | 184.634 | 20.220 |
| AUCINF | 22 | 977.985 | 191.474 | 19.578 |
| CMAX | 22 | 46.155 | 11.430 | 24.764 |
| TMAX | 22 | 11.909 | 6.148 | 51.626 |
| KELM | 22 | 0.046 | 0.010 | 21.274 |
| THALF | 22 | 15.839 | 3.344 | 21.111 |
| TLAG | 22 | 9.545 | 5.031 | 52.702 |
| LAUCTLQC | 22 | 6.798 | 0.201 | 2.952 |
| LAUCINF | 22 | 6.867 | 0.196 | 2.861 |
| LCMAX | 22 | 3.804 | 0.242 | 6.352 |

The results of this study indicate that the test product is bioequivalent to the reference product.

### EXAMPLE 15

In Example 15, a randomized, single-dose, open label two-way crossover study design was used to evaluate the relative bioavailability of a 250 mg test divalproex sodium formulation prepared according to Example 11, to an equivalent oral dose of the commercially available 250 mg delayed release divalproex sodium (Depakote®, Abbott Laboratories, Lot No. 44-228-AA-21, exp. date Sep. 1, 2001), in a test population of 30 healthy adult males under fasting conditions. Sampling times were at hour 0 (within 60 minutes prior to dose), and at post-dosing hours 1, 1.5, 2, 3, 4, 5, 6, 8, 10, 12, 16, 24, 36, 48, 60 and 72. A total of 17 samples per subject were collected in each study period. All subjects consumed 240 ml (8 fl. ozs.) of room temperature tap water simultaneously, at 1.5 hours prior to each dosing, and 120 ml (4 fl. ozs.) of room temperature tap water at post-dose hours 1, 2, and 3, after each dose.

Pharmacokinetic parameters were determined according to the procedure in Example 13. All concentrations are reported as mcg/ml.

The mean plasma valproic acid concentration (mcg/ml) by time point values for the 250 mg test product are listed in Table 12 below:

TABLE 12

| Variable | Time | N | Mean | Std Dev | CV |
|---|---|---|---|---|---|
| C1 | 0.00 HR | 30 | 0.000 | 0.000 | . |
| C2 | 1.00 HR | 30 | 1.896 | 6.337 | 334.264 |
| C3 | 1.50 HR | 30 | 8.014 | 12.102 | 151.013 |
| C4 | 2.00 HR | 30 | 18.034 | 11.489 | 63.707 |
| C5 | 3.00 HR | 30 | 23.559 | 7.579 | 32.171 |
| C6 | 4.00 HR | 30 | 23.903 | 4.320 | 18.073 |
| C7 | 5.00 HR | 30 | 22.063 | 3.874 | 17.560 |
| C8 | 6.00 HR | 30 | 20.137 | 3.447 | 17.120 |
| C9 | 8.00 HR | 30 | 17.067 | 2.878 | 16.862 |
| C10 | 10.0 HR | 30 | 15.040 | 2.769 | 18.413 |
| C11 | 12.0 HR | 30 | 13.147 | 2.291 | 17.425 |
| C12 | 16.0 HR | 30 | 10.464 | 2.201 | 21.031 |
| C13 | 24.0 HR | 30 | 6.981 | 1.752 | 25.100 |
| C14 | 36.0 HR | 30 | 4.047 | 1.220 | 30.153 |
| C15 | 48.0 HR | 30 | 1.690 | 1.521 | 89.993 |
| C16 | 60.0 HR | 30 | 0.572 | 1.089 | 190.432 |
| C17 | 72.0 HR | 30 | 0.086 | 0.473 | 547.723 |

The mean plasma valproic acid concentration (mcg/ml) by time point values for the 250 mg reference product are listed in Table 13 below:

TABLE 13

| Variable | Time | N | Mean | Std Dev | CV |
|---|---|---|---|---|---|
| C1 | 0.00 HR | 30 | 0.000 | 0.000 | . |
| C2 | 1.00 HR | 30 | 0.151 | 0.825 | 547.723 |
| C3 | 1.50 HR | 30 | 1.080 | 5.080 | 470.249 |
| C4 | 2.00 HR | 30 | 5.127 | 7.516 | 146.586 |
| C5 | 3.00 HR | 30 | 18.494 | 11.308 | 61.145 |
| C6 | 4.00 HR | 30 | 22.714 | 7.932 | 34.921 |
| C7 | 5.00 HR | 30 | 22.993 | 3.553 | 15.450 |
| C8 | 6.00 HR | 30 | 21.037 | 3.493 | 16.606 |
| C9 | 8.00 HR | 30 | 17.697 | 2.487 | 14.052 |
| C10 | 10.0 HR | 30 | 15.837 | 2.262 | 14.286 |
| C11 | 12.0 HR | 30 | 13.709 | 2.030 | 14.810 |
| C12 | 16.0 HR | 30 | 10.694 | 1.715 | 16.040 |
| C13 | 24.0 HR | 30 | 7.166 | 1.493 | 20.834 |
| C14 | 36.0 HR | 30 | 4.150 | 1.157 | 27.885 |
| C15 | 48.0 HR | 30 | 1.864 | 1.461 | 78.386 |
| C16 | 60.0 HR | 30 | 0.397 | 0.907 | 228.633 |
| C17 | 72.0 HR | 29 | 0.081 | 0.438 | 538.516 |

The mean (arithmetic) pharmacokinetic values for the 250 mg test product are listed in Table 14 below:

TABLE 14

| Variable | N | Mean | Std Dev | CV |
|---|---|---|---|---|
| AUCTLQC | 30 | 416.079 | 92.598 | 22.255 |
| AUCINF | 30 | 471.240 | 100.583 | 21.344 |
| CMAX | 30 | 27.850 | 4.720 | 16.949 |
| TMAX | 30 | 2.617 | 0.817 | 31.210 |
| KELM | 30 | 0.050 | 0.009 | 18.460 |
| THALF | 30 | 14.437 | 2.835 | 19.639 |
| LAUCTLQC | 30 | 6.008 | 0.218 | 3.628 |
| LAUCINF | 30 | 6.134 | 0.207 | 3.372 |
| LCMAX | 30 | 3.313 | 0.173 | 5.228 |

The mean (arithmetic) pharmacokinetic values for the 250 mg reference product are listed in Table 15 below:

TABLE 15

| Variable | N | Mean | Std Dev | CV |
|---|---|---|---|---|
| AUCTLQC | 30 | 406.296 | 82.077 | 20.201 |
| AUCINF | 30 | 460.435 | 84.942 | 18.448 |
| CMAX | 30 | 26.537 | 4.271 | 16.096 |
| TMAX | 30 | 3.617 | 0.887 | 24.539 |
| KELM | 30 | 0.050 | 0.009 | 17.003 |
| THALF | 30 | 14.228 | 2.491 | 17.506 |
| LAUCTLQC | 30 | 5.988 | 0.200 | 3.334 |
| LAUCINF | 30 | 6.116 | 0.181 | 2.963 |
| LCMAX | 30 | 3.266 | 0.164 | 5.034 |

The results of this study indicate that the test product is bioequivalent to the reference product.

### EXAMPLE 16

In Example 16, a randomized, single-dose, open label, two-way crossover study design was used to evaluate the relative bioavailability of a 125 mg test divalproex sodium formulation prepared according to Example 12, to an equivalent oral dose of the commercially available 125 mg delayed release divalproex sodium (Depakote®, Abbott Laboratories, Lot No. 43-099-AA-22, exp. date Aug. 1, 2001), in a test population of 30 healthy adult males under fasting conditions. Sampling times were at hour 0 (within 60 minutes prior to dose), and at post-dosing hours 1, 1.5, 2, 3, 4, 5, 6, 8, 10, 12, 16, 24, 36, 48, 60 and 72. A total of 17 samples per subject were collected in each study period. All subjects consumed 240 ml (8 fl. ozs.) of room temperature tap water simultaneously at 1.5 hours prior to each dosing, and 120 ml (4 fl. ozs.) of room temperature tap water at post-dose hours 1, 2, and 3, after each dose. Pharmacokinetic parameters were determined according to the procedure in Example 13. All concentrations are reported as mcg/ml.

The mean plasma valproic acid concentration (mcg/ml) by time point values for the 125 mg test product are listed in Table 16 below:

TABLE 16

| Variable | Time | N | Mean | Std Dev | CV |
|---|---|---|---|---|---|
| C1 | 0.00 HR | 28 | 0.000 | 0.000 | . |
| C2 | 1.00 HR | 28 | 0.000 | 0.000 | . |
| C3 | 1.50 HR | 28 | 0.363 | 1.440 | 396.437 |
| C4 | 2.00 HR | 28 | 3.953 | 5.461 | 138.144 |
| C5 | 3.00 HR | 28 | 9.010 | 5.643 | 62.625 |
| C6 | 4.00 HR | 28 | 11.519 | 2.715 | 23.574 |
| C7 | 5.00 HR | 28 | 10.831 | 1.227 | 11.328 |
| C8 | 6.00 HR | 28 | 9.928 | 1.219 | 12.276 |
| C9 | 8.00 HR | 28 | 8.315 | 1.042 | 12.536 |
| C10 | 10.0 HR | 28 | 7.558 | 1.176 | 15.559 |
| C11 | 12.0 HR | 28 | 6.771 | 1.150 | 16.988 |
| C12 | 16.0 HR | 28 | 5.405 | 1.081 | 19.993 |
| C13 | 24.0 HR | 28 | 3.687 | 0.923 | 25.029 |
| C14 | 36.0 HR | 28 | 1.768 | 1.351 | 76.408 |
| C15 | 48.0 HR | 28 | 0.129 | 0.684 | 529.150 |
| C16 | 60.0 HR | 28 | 0.089 | 0.472 | 529.150 |
| C17 | 72.0 HR | 28 | 0.000 | 0.000 | . |

The mean values plasma valproic acid concentration (mcg/ml) by time point for the 125 mg reference product are listed in Table 17 below:

TABLE 17

| Variable | Time | N | Mean | Std Dev | CV |
|---|---|---|---|---|---|
| C1 | 0.00 HR | 28 | 0.000 | 0.000 | . |
| C2 | 1.00 HR | 28 | 0.709 | 3.070 | 432.773 |
| C3 | 1.50 HR | 28 | 2.240 | 4.482 | 200.085 |
| C4 | 2.00 HR | 28 | 5.220 | 5.485 | 105.076 |
| C5 | 3.00 HR | 28 | 10.204 | 3.905 | 38.266 |
| C6 | 4.00 HR | 28 | 10.891 | 2.636 | 24.204 |
| C7 | 5.00 HR | 28 | 10.001 | 1.692 | 16.915 |
| C8 | 6.00 HR | 28 | 9.388 | 1.172 | 12.489 |
| C9 | 8.00 HR | 28 | 8.134 | 1.065 | 13.093 |
| C10 | 10.0 HR | 28 | 7.340 | 1.217 | 16.575 |
| C11 | 12.0 HR | 28 | 6.689 | 1.173 | 17.532 |
| C12 | 16.0 HR | 28 | 5.381 | 1.200 | 22.307 |
| C13 | 24.0 HR | 28 | 3.637 | 1.240 | 34.096 |
| C14 | 36.0 HR | 28 | 1.615 | 1.392 | 86.154 |
| C15 | 48.0 HR | 27 | 0.160 | 0.829 | 519.615 |
| C16 | 60.0 HR | 28 | 0.107 | 0.567 | 529.150 |
| C17 | 72.0 HR | 28 | 0.000 | 0.000 | . |

The mean (arithmetic) pharmacokinetic values for the 125 mg test product are listed in Table 18 below:

TABLE 18

| Variable | N | Mean | Std Dev | CV |
|---|---|---|---|---|
| AUCTLQC | 28 | 179.342 | 51.460 | 28.694 |
| AUCINF | 28 | 235.008 | 63.488 | 27.015 |
| CMAX | 28 | 12.930 | 1.442 | 11.150 |
| TMAX | 28 | 3.250 | 0.752 | 23.124 |
| KELM | 28 | 0.048 | 0.010 | 20.457 |
| THALF | 28 | 15.081 | 3.571 | 23.676 |
| LAUCTLQC | 28 | 5.153 | 0.273 | 5.298 |
| LAUCINF | 28 | 5.428 | 0.252 | 4.636 |
| LCMAX | 28 | 2.554 | 0.112 | 4.375 |

The mean (arithmetic) pharmacokinetic values for the 125 mg reference product are listed in Table 19 below:

TABLE 19

| Variable | N | Mean | Std Dev | CV |
|---|---|---|---|---|
| AUCTLQC | 28 | 176.864 | 58.355 | 32.994 |
| AUCINF | 28 | 235.357 | 72.420 | 30.770 |
| CMAX | 28 | 12.424 | 1.922 | 15.467 |
| TMAX | 28 | 3.273 | 1.164 | 35.561 |
| KELM | 28 | 0.049 | 0.012 | 23.995 |
| THALF | 28 | 14.987 | 3.744 | 24.985 |
| LAUCTLQC | 28 | 5.130 | 0.303 | 5.915 |
| LAUCINF | 28 | 5.423 | 0.274 | 5.047 |
| LCMAX | 28 | 2.508 | 0.161 | 6.411 |

The results of this study indicate that the test product is bioequivalent to the reference product.

What is claimed is:

1. An oral solid dosage form comprising a therapeutically effective amount of neutralized divalproex sodium which provides a delayed release of valproate ion when the neutralized divalproex sodium dosage form is orally administered to human patients, said dosage form being bioavailable and providing a therapeutic effect which is bioequivalent to a delayed release divalproex sodium tablet, (Depakote®).

2. The dosage form of claim 1, which provides a mean time to maximum plasma concentration (TMAX) of valproic acid at from about 1.8 to about 13.15 hours after oral administration.

3. The dosage form of claim 1, which provides a mean time to maximum plasma concentration (TMAX) of valproic acid at from about 2.7 to about 13.15 hours after oral administration.

4. The dosage form of claim 1, which provides a mean time to maximum plasma concentration (TMAX) of valproic acid at from about 2.7 to about 4.8 hours after oral administration in the fasted state.

5. The dosage form of claim 1, which provides a mean time to maximum plasma concentration (TMAX) of valproic acid at from about 5 to about 13.15 hours after oral administration in the fed state.

6. The dosage form of claim 1, which provides a mean time to maximum plasma concentration (TMAX) of valproic acid at from about 1.8 to about 3.5 hours after oral administration.

7. The dosage form of claim 1, which provides a mean time to maximum plasma concentration (TMAX) of valproic acid at from about 2.4 to about 4 hours after oral administration.

8. The dosage form of claim 1, which provides a mean maximum plasma concentration (CMAX) of valproic acid of from about 38 to about 67 mcg/ml, based on oral administration of a 500 mg delayed release dose of neutralized divalproex sodium.

9. The dosage form of claim 1, which provides a mean maximum plasma concentration (CMAX) of valproic acid of from about 46 to about 67 mcg/ml, based on oral administration in the fasted state of a 500 mg delayed release dose of neutralized divalproex sodium.

10. The dosage form of claim 1, which provides a mean maximum plasma concentration (CMAX) of valproic acid of from about 38 to about 59 mcg/ml, based on oral administration in the fed state of a 500 mg delayed release dose of neutralized divalproex sodium.

11. The dosage form of claim 1, which provides a mean maximum plasma concentration (CMAX) of valproic acid of from about 23 to about 33 mcg/ml, based on oral administration of a 250 mg delayed release dose of neutralized divalproex sodium.

12. The dosage form of claim 1, which provides a mean maximum plasma concentration (CMAX) of valproic acid of from about 11 to about 15 mcg/ml, based on oral administration of a 125 mg delayed release dose of neutralized divalproex sodium.

13. The dosage form of claim 1, which provides a mean AUC from time zero to last quantifiable concentration (AUCTLQC) of valproic acid of from about 763 to about 1154 mcg·hr/ml, based on oral administration of a 500 mg delayed release dose of neutralized divalproex sodium.

14. The dosage form of claim 1, which provides a mean AUC from time zero to last quantifiable concentration (AUCTLQC) of valproic acid of from about 771 to about 1154 mcg·hr/ml, based on oral administration in the fasted state of a 500 mg delayed release dose of neutralized divalproex sodium.

15. The dosage form of claim 1, which provides a mean AUC from time zero to last quantifiable concentration (AUCTLQC) of valproic acid of from about 763 to about 1087 mcg·hr/ml, based on oral administration in the fed state of a 500 mg delayed release dose of neutralized divalproex sodium.

16. The dosage form of claim 1, which provides a mean AUC from time zero to last quantifiable concentration (AUCTLQC) of valproic acid of from about 323 to about 509 mcg·hr/ml, based on oral administration of a 250 mg delayed release dose of neutralized divalproex sodium.

17. The dosage form of claim 1, which provides a mean AUC from time zero to last quantifiable concentration (AUCTLQC) of valproic acid of from about 127 to about 231 mcg·hr/ml, based on oral administration of a 125 mg delayed release dose of neutralized divalproex sodium.

18. The dosage form of claim 1, which provides a mean AUC from time zero to infinity (AUCINF) of valproic acid of 1062±227 mcg·hr/ml based on oral administration in the fasted state of a 500 mg delayed release dose of neutralized divalproex sodium.

19. The dosage form of claim 1, which provides a mean AUC from time zero to infinity (AUCINF) of valproic acid of 1038±192 mcg·hr/ml based on oral administration in the fasted state of a 500 mg delayed release dose of neutralized divalproex sodium.

20. The dosage form of claim 1, which provides a mean AUC from time zero to infinity (AUCINF) of valproic acid of 988±173 mcg·hr/ml based on oral administration in the fed state of a 500 mg delayed release dose of neutralized divalproex sodium.

21. The dosage form of claim 1, which provides a mean AUC from time zero to infinity (AUCINF) of valproic acid of 471±100 mcg·hr/ml based on oral administration of a 250 mg delayed release dose of neutralized divalproex sodium.

22. The dosage form of claim 1, which provides a mean AUC from time zero to infinity (AUCINF) of valproic acid of 235±63 mcg·hr/ml based on oral administration of a 125 mg delayed release dose of neutralized divalproex sodium.

23. The dosage form of claim 1 which provides a mean AUC to last quantifiable concentration (AUCTLQC) of valproic acid which is from about 80% to about 125% of the AUC to last quantifiable concentration (AUCTLQC) of valproic acid provided by oral administration of a reference standard over the same time interval.

24. The dosage form of claim 1 which provides a mean AUC to last quantifiable concentration (AUCTLQC) of valproic acid which is from about 80% to about 125% of the AUC to last quantifiable concentration (AUCTLQC) of valproic acid provided by oral administration of a reference standard over the same time interval, and wherein the dosage form and the reference standard are orally administered in a fasted state.

25. The dosage form of claim 1 which provides a mean AUC to last quantifiable concentration (AUCTLQC) of valproic acid which is from about 80% to about 125% of the AUC to last quantifiable concentration (AUCTLQC) of valproic acid provided by oral administration of a reference standard over the same time interval, and wherein the dosage form and the reference standard are orally administered in a fed state.

26. The dosage form of claim 1 which provides a mean half-life (THALF) of valproic acid of about 10.9 to about 21.3 hours.

27. An oral solid dosage form comprising a therapeutically effective amount of neutralized divalproex sodium which provides a delayed release of valproate ion when the neutralized divalproex sodium dosage form is orally administered to human patients, said dosage form being bioavailable and providing a therapeutic effect, and said dosage form providing a mean time to maximum plasma concentration (TMAX) of valproic acid at from about 1.8 to about 13.15 hours after oral administration.

28. The dosage form of claim 27, which provides a mean time to maximum plasma concentration (TMAX) of valproic acid at from about 2.7 to about 13.15 hours after oral administration.

29. An oral solid dosage form comprising a therapeutically effective dose of neutralized divalproex sodium and a pharmaceutically acceptable carrier, said dosage form providing a mean time to maximum plasma concentration (TMAX) of valproic acid at from about 1.8 to about 13.15 hours after oral administration.

30. The dosage form of claim 29, which provides a mean time to maximum plasma concentration (TMAX) of valproic acid at from about 2.7 to about 13.15 hours after oral administration.
